(19) United States
(12) Patent Application Publication
(10) Pub. No.: US 2021/0121083 A1
(43) Pub. Date: Apr. 29, 2021

(54) MODEL SETTING DEVICE, CONTACTLESS BLOOD PRESSURE MEASUREMENT DEVICE, MODEL SETTING METHOD, AND RECORDING MEDIUM

(71) Applicant: SHARP KABUSHIKI KAISHA, Sakai City, Osaka (JP)

(72) Inventors: Yuki EDO, Sakai City (JP); Yoshihisa ADACHI, Sakai City (JP); Rieko OGAWA, Sakai City (JP); Ryota TOMIZAWA, Sakai City (JP)

(73) Assignee: Sharp Kabushiki Kaisha, Sakai City, Osaka (JP)

(21) Appl. No.: 17/041,467

(22) PCT Filed: Feb. 22, 2019
(86) PCT No.: PCT/JP2019/006771

§ 371 Date: Sep. 25, 2020

(30) Foreign Application Priority Data
Mar. 27, 2018 (JP) 2018-060592

(51) Int. Cl. A61B 5/021; A61B 5/00; G16H 50/50; G16H 50/30

(52) U.S. Cl. CPC A61B 5/02125; A61B 5/7275; G16H 50/30; G16H 50/50; A61B 5/742

(57) ABSTRACT

A model setting device configured to set a model based on pulse waves at first and second target parts of a living body, and acquire a plurality of combinations of a blood pressure value and a plurality of images of the living body, the model setting device includes: a region setter configured to perform a process of setting a plurality of first target regions in the first target part and setting a plurality of second target regions in the second target part on the plurality of images; a detector configured to detect pulse waves of the living body; a calculator configured to calculate a pulse wave propagation time between the first and second target regions; an evaluator configured to evaluate an accuracy of a blood pressure prediction by using the first and second target regions; and a determiner configured to determine preferable sizes of the first and second target regions.

### TECHNICAL FIELD

The present disclosure relates to model setting devices or the like, and more specifically, to a model setting device for setting a blood pressure prediction model, a contactless blood pressure measurement device using the model, or the like.

### BACKGROUND ART

As a technique for measuring blood pressure of a human body, a contactless blood pressure measurement technique for measuring (estimating) the blood pressure without contacting the human body has been developed recently (see, for example, PTL 1 to PTL 3).

Such a technique predicts blood pressure based on a pulse wave propagation time calculated from pulse waves at two different parts, for example, the forehead and the cheek, of a human body based on an image captured by, for example, a camera.

### CITATION LIST

### Patent Literature

PTL 1: Japanese Unexamined Patent Application Publication No. 2017-104491 (publication date: Jun. 15, 2017)
PTL 2: Japanese Unexamined Patent Application Publication No. 2016-190022 (publication date: Nov. 10, 2016)
PTL 3: Japanese Unexamined Patent Application Publication No. 2015-054223 (publication date: Mar. 23, 2015)

### SUMMARY OF INVENTION

### Technical Problem

For a pulse wave calculation, a technique for defining target regions for respective parts, calculating an average luminance value of a plurality of pixels included in each target region, and calculating a pulse wave based on a change in the average luminance value has been known.

An optimal size of each target region for a blood pressure prediction is, however, not taken into consideration, which leads to the problem that the blood pressure cannot accurately be predicted.

One of objects of the present disclosure is to provide a model setting device or the like configured to improve the prediction accuracy of blood pressure by the blood pressure measurement device by appropriately setting a blood pressure prediction model used in a contactless blood pressure measurement device.

### Solution to Problem

To solve the problem, a model setting device of one aspect of the present disclosure is a model setting device configured to set a model for predicting blood pressure of a living body based on pulse waves at first and second target parts of the living body. The model setting device is configured to acquire a plurality of combinations of a blood pressure value of the living body and a plurality of images of the living body when the blood pressure value is measured. The model setting device includes: a region setter configured to perform a process of setting a plurality of first target regions having different sizes in the first target part and setting a plurality of second target regions having different sizes in the second target part on the plurality of images included in each of the plurality of combinations; a detector configured to detect pulse waves of the living body based on the plurality of first and second target regions thus set; a calculator configured to calculate a pulse wave propagation time between the first and second target regions from the pulse waves thus detected; an evaluator configured to evaluate, based on the blood pressure value and the pulse wave propagation time, an accuracy of a blood pressure prediction by using the first and second target regions for each combination of the sizes of the first and second target regions; and a determiner configured to determine preferable sizes of the first and second target regions based on an evaluation result by the evaluator.

A model setting method of one aspect of the present disclosure is a model setting method for setting a model for predicting blood pressure of a living body based on pulse waves at first and second target parts of the living body. The model setting method includes: a step of acquiring a plurality of combinations of a blood pressure value of the living body and a plurality of images of the living body when the blood pressure value is measured; a step of performing a process of setting a plurality of first target regions having different sizes in the first target part and setting a plurality of second target regions having different sizes in the second target part on the plurality of images included in each of the plurality of combinations; a step of detecting pulse waves of the living body based on the plurality of first and second target regions thus set; a step of calculating a pulse wave propagation time between the first and second target regions from the pulse waves thus detected; a step of evaluating, based on the blood pressure value and the pulse wave propagation time, an accuracy of a blood pressure prediction by using the first and second target regions for each combination of the sizes of the first and second target regions; and a step of determining preferable sizes of the first and second target regions based on the evaluation.

### Advantageous Effects of Invention

One aspect of the present disclosure enables the accuracy of blood pressure prediction to be improved in a contactless blood pressure measurement device.

### BRIEF DESCRIPTION OF DRAWINGS

FIG. 1 is a functional block diagram illustrating a configuration of a blood pressure measurement system according to a first embodiment.

FIG. 2 is a view schematically illustrating a configuration of a camera.

FIG. 3 is a flowchart illustrating an example of a flow of a process in a data acquisition unit and a model setting unit.

FIG. 4 is a view illustrating a setting method of first and second target regions by a region setter.

FIG. 5 is a view illustrating a process of calculating arithmetic values of pixel values of colors in a cheek region of a captured image.

FIG. 6 is a view illustrating a pulse wave propagation time calculated from pulse waves at the cheek region and a forehead region.

FIG. 7 is a view illustrating a relationship between the pulse wave propagation time and a cuff blood-pressure value in the cheek region and the forehead region when the sizes of the cheek region and the forehead region have three stages.

FIG. 8 is a view illustrating a relationship between the number of pixels included in one row of a square of each of the cheek region and the forehead region and an adjusted R square.

FIG. 9 is a view illustrating a method for calculating waveform information from a pulse waveform of the forehead region.

FIG. 10 is a graph illustrating a relationship between a mean squared prediction error and a regularization parameter when Lasso regression is performed.

FIG. 11 is a view illustrating a result of comparison between a case where Lasso regression is performed without using the waveform information and a case where the Lasso regression is performed by using the waveform information.

FIG. 12 is a flowchart illustrating an example of a flow of a process in a data acquisition unit and a model setting unit according to a third embodiment.

### DESCRIPTION OF EMBODIMENTS

To facilitate the understanding of an operation device and the like in the present disclosure, the following description starts with the outline of the knowledge of the present disclosure.

Target regions may be defined for respective parts of a subject, an average luminance value of a plurality of pixels included in each target region may be calculated, and a pulse wave may be calculated based on a change in the average luminance value. In this case, when each target region is too small (when the number of pixels to be averaged is small), noise in a camera signal is great, and a minute pulse wave signal is not accurately obtained.

In contrast, when each target region is too large (when the number of pixels to be averaged is large), the influence of noise over the camera signal is small, but a region in which detection of a pulse wave is difficult, for example, a region in which blood vessels are located at a lower part of the epidermis is highly possibly used, and therefore, a pulse wave propagation time unsuitable for a blood pressure prediction may be calculated.

Moreover, it is expected that an optimal target region varies between individuals, between parts, and between measured parts. Optimizing the size of the target region for each individual and/or each measured part enables a pulse wave propagation time to be calculated with high accuracy of the blood pressure prediction.

### First Embodiment

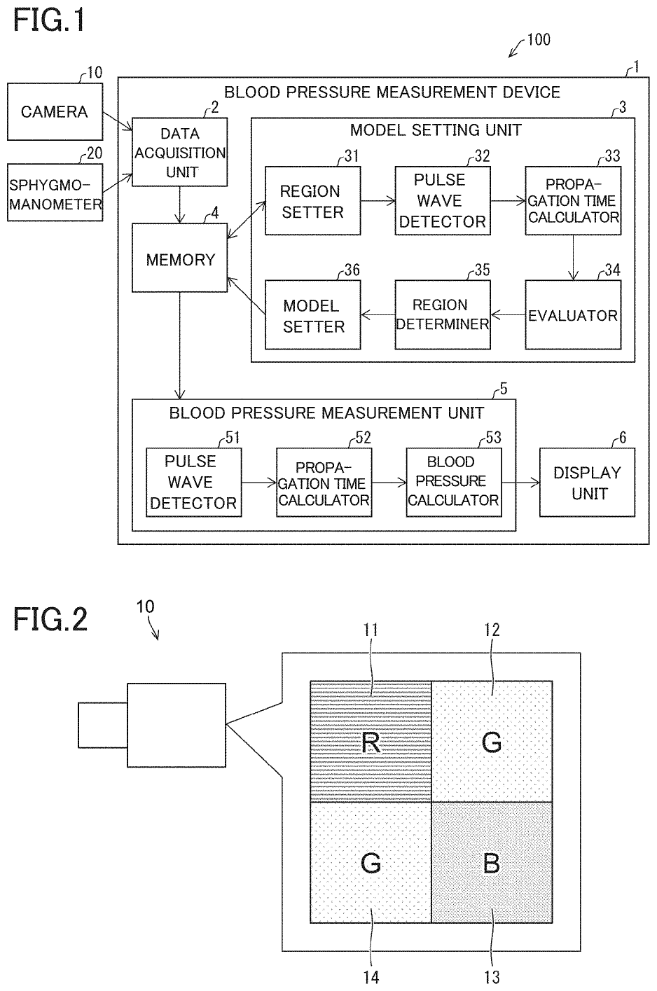

One embodiment of the present invention will be described in detail below. FIG. 1 is a functional block diagram illustrating a configuration of a blood pressure measurement system 100 according to the present embodiment. The blood pressure measurement system 100 is a system including a blood pressure measurement device 1 (contactless blood pressure measurement device) configured to predict the blood pressure of a subject (living body) based on pulse waves at two different target parts of the subject. As illustrated in FIG. 1, the blood pressure measurement system 100 includes the blood pressure measurement device 1, a camera 10, and a sphygmomanometer 20.

The camera 10 includes an image sensor (not shown) having a plurality of light receiving elements. The camera 10 captures an image of the subject a plurality of number of times at prescribed time intervals (e.g., a frame rate is 300 fps) to generate a plurality of captured images and transmits the plurality of captured images to the blood pressure measurement device 1. In the following description, the camera 10 transmits a moving image including the plurality of captured images to the blood pressure measurement device 1. Note that the camera 10 does not have to be communicatively connected to the blood pressure measurement device 1, but a storage medium storing the moving image may be inserted into or connected to the blood pressure measurement device 1 to provide the blood pressure measurement device 1 with the moving image.

FIG. 2 is a view schematically illustrating a configuration of the camera 10. As illustrated in FIG. 2, the respective light receiving elements included in the image sensor (not shown) of the camera 10 are provided with a red filter 11, a first green filter 12, a blue filter 13, and a second green filter 14. The second green filter 14 transmits green light within a visible light wavelength range from about 500 nm to about 600 nm in a similar manner to the first green filter 12. The second green filter 14 also transmits light within a near infrared wavelength range of about 805 nm or longer.

The camera 10 detects respective intensities (luminances) of light transmitted through the red filter 11, the first green filter 12, the blue filter 13, and the second green filter 14 and generates a captured image. Each light receiving element provided with any one of the above-described four kinds of filters forms a corresponding one of pixels in the captured image.

The camera 10 generates a moving image by capturing the subject based on the intensities of respective light transmitted through the red filter 11, the first green filter 12, the blue filter 13, and the second green filter 14, and outputs the moving image thus generated to the blood pressure measurement device 1.

Note that the camera 10 may include an infrared light filter which transmits light within a near infrared wavelength range of about 805 nm or longer in place of the second green filter 14. Alternatively, the camera 10 may include filters of three colors, namely, the red filter 11, the first green filter 12, and the blue filter 13.

The sphygmomanometer 20 is a contact-type sphygmomanometer for measuring the blood pressure of the subject and is, for example, a cuff sphygmomanometer. The sphygmomanometer 20 is communicatively connected to the blood pressure measurement device 1. A blood pressure value measured by the sphygmomanometer 20 is transmitted to the data acquisition unit 2 of the blood pressure measurement device 1. Note that a user may input the blood pressure value measured by the sphygmomanometer 20 to the blood pressure measurement device 1 via an input unit (not shown) of the blood pressure measurement device 1.

While the camera 10 captures a moving image, the blood pressure has to be measured with the sphygmomanometer 20. Thus, the blood pressure measurement device 1 controls the camera 10 and the sphygmomanometer 20 such that capturing of images by the camera 10 and measurement by the sphygmomanometer 20 are performed simultaneously.

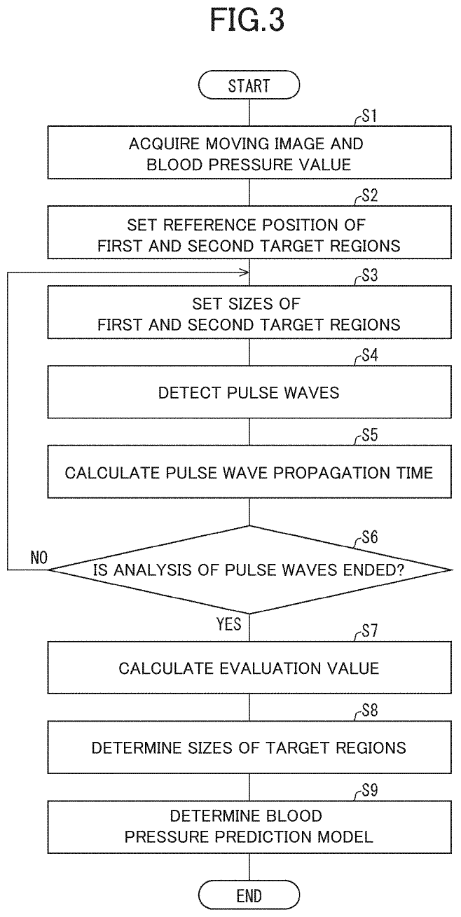

As illustrated in FIG. 1, the blood pressure measurement device 1 includes the data acquisition unit 2, a model setting unit 3 (model setting device), memory 4, a blood pressure measurement unit 5, and a display unit 6. FIG. 3 is a flowchart illustrating an example of a flow of a process in the data acquisition unit 2 and the model setting unit 3.

The data acquisition unit 2 receives the blood pressure value (blood pressure value data) of the subject from the sphygmomanometer 20, receives the moving image (moving image data) of the subject from the camera 10, and stores, in the memory 4, the blood pressure value and the moving image thus received (S1). The memory 4 is a non-volatile storage device.

The blood pressure value data and the moving image data are associated with each other, and a combination of these pieces of data corresponds to a combination of the blood pressure value of the subject and the plurality of images of the subject when the blood pressure value is measured. The moving image data is, for example, data of a moving image obtained by capturing the face of the subject for 60 seconds. The blood pressure value data is data representing a blood pressure value obtained by measuring the subject with the sphygmomanometer 20 while the moving image is captured (for the 60 seconds). Such a combination of the blood pressure value data and the moving image data is referred to as a data set.

The data acquisition unit 2 acquires a plurality of data sets having different blood pressure values. In the present embodiment, the data acquisition unit 2 acquires 14 data sets. Of these data sets, a first data set includes a first moving image and a first blood pressure value, and an nth data set includes an nth moving image and an nth blood pressure value. The moving image data included in the data set is, for example, a 60-second moving image of face (18000 frames in total) including 640 columns and 480 rows of pixels. The data acquisition unit 2 may be provided to each of the model setting unit 3 and the blood pressure measurement unit 5.

The blood pressure of the subject is variable by, for example, aerobike (registered trademark) exercise (load: 50 W to 80 W). The data acquisition unit 2 acquires a data set while the subject is resting and a plurality of data sets when the blood pressure varies. Note that in the present embodiment, the condition is that the face of the subject is fixed such that the face does not move with respect to the camera 10, and the face hardly moves in all of the 14 moving images.

As illustrated in FIG. 1, the model setting unit 3 includes a region setter 31, a pulse wave detector 32, a propagation time calculator 33, an evaluator 34, a region determiner 35, and a model setter 36.

The region setter 31 sets, in each of the captured images included in the moving image of the subject stored in the memory 4, a region (a target region) serving as a target at which a pulse wave is to be detected. Specifically, the region setter 31 sets a plurality of first target regions having different sizes for one of the two target parts in a face region 80 and a plurality of second target regions having different sizes for the other of the two target parts. Note that the first and second target regions have to be selected from regions which are included in the captured image and in which the skin of the subject is captured. This is because the pulse wave is detected with reference to a temporal change in color of the skin of the subject.

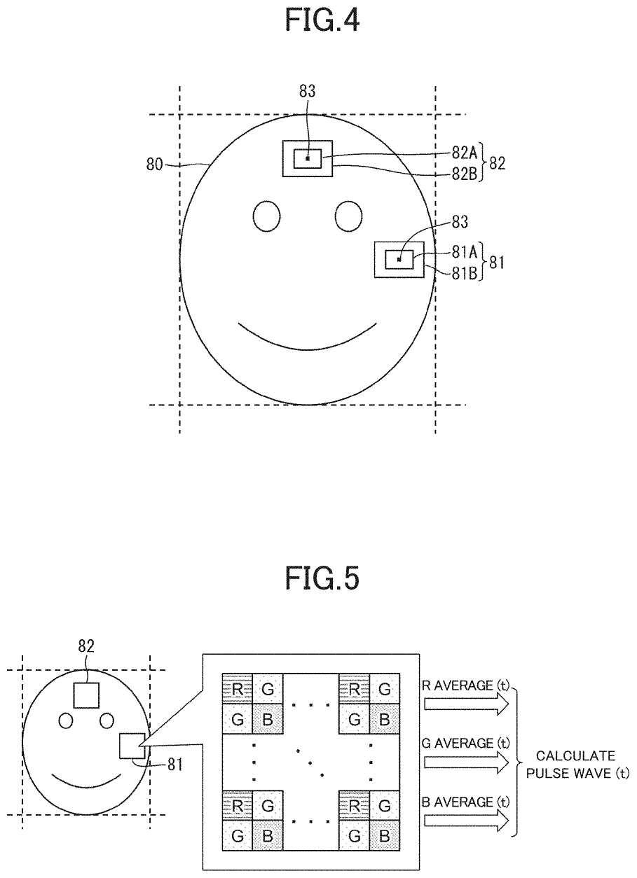

FIG. 4 is a view illustrating a setting method of the first and second target regions by the region setter 31. As illustrated in FIG. 4, the region setter 31 detects the face region 80 of the subject in each of the prescribed frames of the moving image of the subject and sets reference positions of the first and second target regions in the face region 80 (S2 in FIG. 3). The first and second target regions are set in two different target parts included in the face region 80. For detection of the face region 80, a publicly known technique may be used.

In the present embodiment, for example, the region setter 31 sets the first target region (a cheek region 81) in the cheek (a first target part) and sets the second target region (a forehead region 82) in the forehead (a second target part). As the target region, a nose region may be set in place of the cheek region 81 or the forehead region 82. The forehead, nose, and cheek have arteries, and in addition, the forehead, nose, and cheek are regions in which detection is easily performed when the face of the subject faces the camera 10, and therefore, the forehead, nose, and cheek are preferable as target regions. Moreover, when the subject faces sideways with respect to the camera 10, the neck may be the target region.

Then, the region setter 31 sets a plurality of target regions having different sizes for each of the cheek region 81 and the forehead region 82 (S3 of FIG. 3). FIG. 4 shows cheek regions 81A⋅81B set for the cheek region 81 and forehead regions 82A⋅82B set for the forehead region 82. As described above, the region setter 31 sets a plurality of the first target regions having different sizes and the second target regions having different sizes for the respective two target parts (cheek and forehead).

The sizes of the cheek region 81 and the forehead region 82 are, for example, changed in the range of 2×2 pixels to 100×100 pixels. The upper limit of the sizes is at least set such that the cheek region 81 and the forehead region 82 do not largely extend beyond the cheek part and the forehead part, respectively.

The number of sizes of the first and second target regions is not particularly limited. For simplification of the description, two target regions having different sizes are set for the cheek region 81 and two target regions having different sizes are set for the forehead region 82 in the following description.

Note that in the following description, simply denoting the cheek region 81 collectively means the cheek regions 81A⋅81B having different sizes, and simply denoting the forehead region 82 collectively means the forehead regions 82A⋅82B.

In order to set the cheek regions 81 having different sizes, the region setter 31 may set the cheek region 81 (cheek region 81B) having a size changed to share, as the reference position, a center 83 of the cheek region 81A (reference first target region) having a reference size. Alternatively, the region setter 31 may set the cheek region 81 (cheek region 81B) having a size changed to share, as the reference position, one of apexes of the cheek region 81A and to include the cheek region 81A (or to be included in the cheek region 81A). This also applies to the forehead region 82.

The region setter 31 sets the cheek region 81 and the forehead region 82 for each of the plurality of captured images included in the first to fourteenth moving images. The region setter 31 stores, in the memory 4, information representing the locations and the sizes of the cheek region 81 and the forehead region 82 thus set.

The pulse wave detector 32 calculates (S4 of FIG. 3) the pulse wave of the subject by detecting a change in average pixel value of each color in each of the cheek regions 81A⋅B and the forehead regions 82A⋅B located at the locations and having the sizes set by the region setter 31. The pulse wave detector 32 computes the average pixel value for each of the plurality of captured images included in the first to fourteenth moving images and detects pulse waves for each moving image and for each of the cheek regions 81A⋅81B, and for each moving image and for each of the forehead regions 82A⋅82B.

FIG. 5 is a view illustrating a process of calculating arithmetic values of pixel values of colors in the cheek region 81 of a captured image. As illustrated in FIG. 5, pixels included in the cheek region 81 and the forehead region 82 in the captured image are arranged in an RGB Bayer pattern. The pulse wave detector 32 calculates arithmetic values of pixel values of colors in the cheek region 81 and the forehead region 82 by using pixel values (gray scale values) of colors (R, G, B) included in the cheek region 81 and the forehead region 82. The arithmetic values are values reflecting the sizes of the pixel values of the pixels included in the cheek region 81 and the forehead region 82.

The pulse wave detector 32 may calculate the average of pixel values (average pixel value) of the colors in, for example, the cheek region 81 as an arithmetic value of the pixel values in the cheek region 81. Moreover, the pulse wave detector 32 may calculate, for example, a statistical value as the arithmetic value of the pixel values in the cheek region 81. The statistical value is calculated with weighting on the pixel values of pixels close to the center of the cheek region 81 being increased and weighting of the pixel values of pixels away from the center of the cheek region 81 being reduced. In the following description, it is assumed that the pulse wave detector 32 calculates the average pixel value of each color in the cheek region 81 as the arithmetic value of the pixel value in the cheek region 81. The same applies to the forehead region 82. Note that of pixels in the cheek region 81 and the forehead region 82, pixels having a luminance value lower than or equal to a predetermined value do not have to be used.

In order to acquire a temporal change in the average pixel value, the pulse wave detector 32 calculates the average pixel value for a frame corresponding to a prescribed time (e.g., for 30 seconds) in the moving image.

The pulse wave detector 32 detects a change in the average pixel value of each color to calculate the pulse wave of the subject for each moving image and for each of the cheek region 81 or the forehead region 82. That is, the pulse wave detector 32 performs detection of the pulse wave based on the cheek region 81A in the first moving image, detection of the pulse wave based on the cheek region 81B in the first moving image, detection of the pulse wave based on the forehead region 82A in the first moving image, and detection of the pulse wave based on the forehead region 82B in the first moving image. The pulse wave detector 32 performs the detection of such pulse waves on each of the 14 moving images.

Specifically, the pulse wave detector 32 first performs an independent component analysis on the average pixel value of each color and extracts the same number of (that is, three) independent components as the number of colors. The pulse wave detector 32 removes, from the three independent components extracted, a low-frequency component and a high frequency component with a digital bandpass filter of 0.75 to 3.0 Hz.

Then, the pulse wave detector 32 performs fast Fourier transformation on the three independent components, from which the low-frequency component and the high frequency component have been removed, and calculates a power spectrum of a frequency of each independent component. The pulse wave detector 32 calculates a peak value in a range from 0.75 to 3.0 Hz of the power spectrum of the frequency of each independent component thus calculated and detects, as a pulse wave, the independent component having a peak having the largest peak value of the peak values of the independent components. The pulse wave detector 32 outputs the pulse wave thus detected to the propagation time calculator 33.

Note that when a variation in the average pixel value is large with respect to time, the pulse wave detector 32 may perform detrending on the average pixel value of each color (see IEEE Trans Biomed Eng, 2002 February; 49(2):172-175) and may perform an independent component analysis on the average pixel value of each color, from which the variation has been removed.

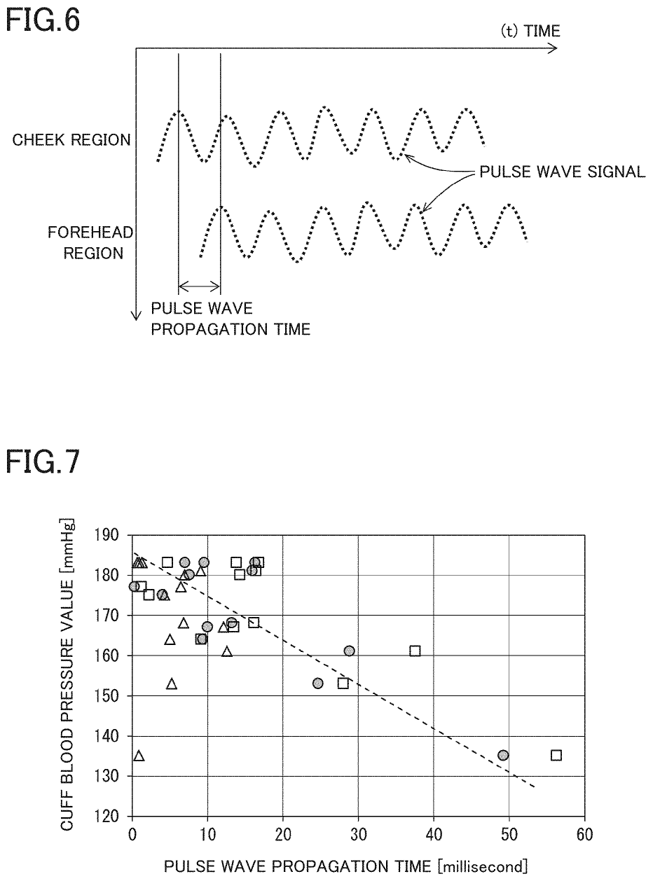

FIG. 6 is a view illustrating a pulse wave propagation time calculated from pulse waves at the cheek region 81 and the forehead region 82. As illustrated in FIG. 6, the propagation time calculator 33 calculates, as the pulse wave propagation time, a time difference between the pulse wave at the forehead region 82 and the pulse wave at the cheek region 81 as the reference (S5 in FIG. 3). The pulse wave arrives early at the cheek close to the heart, and therefore, the time difference usually has a plus sign.

As the computation method of the pulse wave propagation time, a method such as a cross-correlation analysis is used. For example, a correlation coefficient between the pulse waves when the pulse wave at the forehead region 82 is continuously shifted by a slight time difference with the cheek region 81 as the reference is obtained, and a time difference when the correlation coefficient is largest is calculated as the pulse wave propagation time of the two pulse waves.

The pulse wave detector 32 performs the calculation of such a pulse wave propagation time on a combination of one of the cheek regions 81A⋅B and one of the forehead regions 82A⋅B of a moving image. Thus, four combinations are obtained for one moving image. The pulse wave detector 32 calculates the pulse wave propagation time for all or some of the combinations. The pulse wave detector 32 performs such processes on each of the fourteen moving images (YES in S6 of FIG. 3) and outputs a result of the processes to the evaluator 34.

FIG. 7 shows a relationship between the pulse wave propagation time in the cheek region 81 and the forehead region 82 and a cuff blood-pressure value when the cheek region 81 and the forehead region 82 each have three sizes (8×8, 16×16, 32×32). Circular symbols represent values when the size of the cheek region 81 is 32×32, and the size of the forehead region 82 is 16×16. The adjusted R square in this case is 0.79. Quadrangular symbols represent values when the size of the cheek region 81 is 8×8, and the size of the forehead region 82 is 16×16. The adjusted R square in this case is 0.60. Triangular symbols represent values when the size of the cheek region 81 is 32×32, and the size of the forehead region 82 is 32×32. The adjusted R square in this case is 0.01.

The broken line of the graph in FIG. 7 represents a regression formula of the pulse wave propagation time and the cuff blood-pressure values corresponding to the circular symbols. When such a linear or near-linear relationship is obtained, assuming a linear model “(cuff blood-pressure values)=(segment)+(gradient)×(pulse wave propagation time)” is suitable, and calculating the segment and the gradient from the regression formula enables a preferable blood pressure prediction model to be calculated.

As the adjusted R square increases, the regression formula is more adapted to data, and therefore, it can be said that the broken line is a blood pressure prediction model obtained when the sizes of the cheek region 81 and the forehead region 82 are optimized.

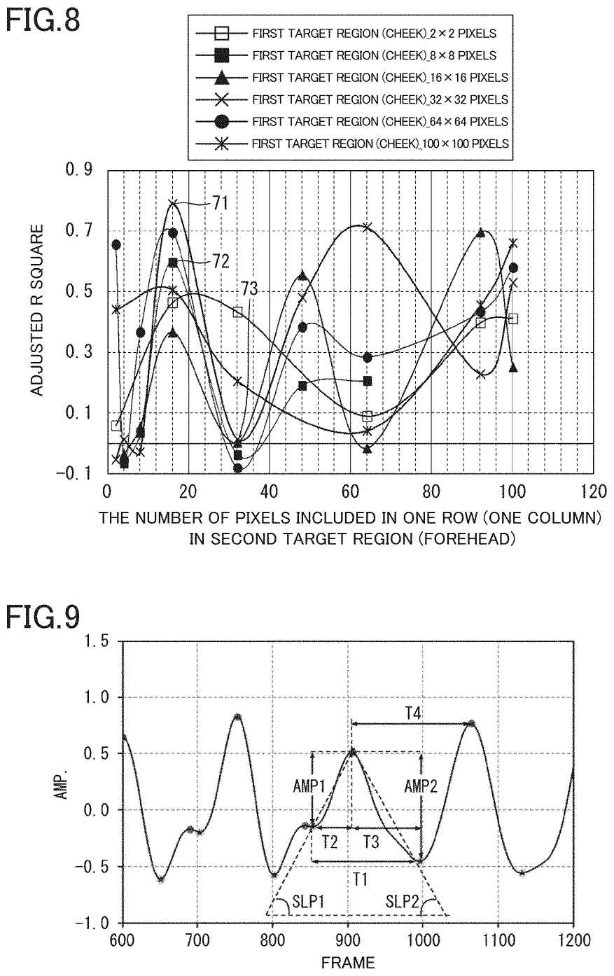

FIG. 8 shows a relationship between the number of pixels included in one row (one column) of a square of each of the cheek region 81 and the forehead region 82 and an adjusted R square. FIG. 8 shows a result obtained by changing the size of each of the cheek region 81 and the forehead region 82 to a size of 2×2 pixels to 100×100 pixels and by performing, for a total of 36 combinations of region sizes, estimation of the regression formula by least square assuming the linear model, where the blood pressure is defined as an object variable and the pulse wave propagation time is defined as an explanatory variable.

As illustrated in FIG. 8, when the cheek region 81 has 32×32 pixels, and the forehead region 82 has 16×16 pixels (in the case denoted by reference number 71), the adjusted R square is 0.79 and which is largest. The circular symbols in FIG. 7 correspond to the symbols denoted by reference number 71 in FIG. 8. The quadrangular symbols in FIG. 7 correspond to the symbols denoted by reference number 72 in FIG. 8. The triangular symbols in FIG. 7 correspond to the symbols denoted by reference number 73 in FIG. 8.

Thus, blood pressure prediction performance significantly depends on the size (the number of pixels to be averaged) of the target region. Moreover, when the cheek region 81 and the forehead region 82 each have 100×100 pixels, these regions extend beyond the cheek and the forehead respectively. Therefore, even when the cheek region 81 and the forehead region 82 are enlarged to have 100×100 pixels or more, performance better than that obtained by the optimal size cannot probably be expected.

Based on such views, the evaluator 34 evaluates the accuracy of the blood pressure prediction for each combination of the sizes of the cheek region 81 and the forehead region 82. Specifically, the evaluator 34 evaluates to which degree the relationship between the blood pressure value of a subject and a pulse wave propagation time between the cheek region 81 and the forehead region 82 of the subject when the blood pressure value is measured approximates the prescribed relationship. For example, the evaluator 34 performs the estimation of the regression formula by the least square assuming the linear model, where the blood pressure of the subject is defined as an object variable, and the pulse wave propagation time is defined as an explanatory variable, thereby calculating the adjusted R square as an evaluation value representing the accuracy of the blood pressure predication (S7 in FIG. 3).

The region determiner 35 determines preferable sizes of the cheek region 81 and the forehead region 82 based on the evaluation result by the evaluator 34. Specifically, the region determiner 35 determines “a combination of the size of the cheek region 81 and the size of the forehead region 82” whose adjusted R square is largest to be a combination of preferable sizes of the cheek region 81 and the forehead region 82 (S8 of FIG. 3).

As a performance index of the blood pressure prediction model, Akaike's Information Criteria (AIC), or mean squared error to which unknown data is applied, or the like other than the adjusted R square may be used. However, the mean squared error whose statistical handling by a method such as cross validation or the like is easy is most preferably used as the index.

The model setter 36 sets a model corresponding to a combination of the sizes of the cheek region 81 and the forehead region 82 determined by the region determiner 35 as the model for predicting the blood pressure (S9 of FIG. 3). That is, the model setter 36 determines, as a model to be used by the model setting unit 3, a model having the highest predicting performance (largest evaluation value) of models created. The model setter 36 stores the set model in the memory 4.

The blood pressure measurement unit 5 analyzes a captured image of a subject captured by the camera 10 to measure the blood pressure of the subject. The blood pressure measurement unit 5 measures the blood pressure of the subject based on the model set by the model setter 36.

The blood pressure measurement unit 5 includes a pulse wave detector 51, a propagation time calculator 52, and a blood pressure calculator 53. The pulse wave detector 51 performs a process similar to that performed by the pulse wave detector 32, thereby detecting pulse waves at the cheek region 81 and the forehead region 82. The propagation time calculator 52 performs a process similar to that performed by the propagation time calculator 33, thereby calculating the pulse wave propagation time between the cheek region 81 and the forehead region 82. The sizes of the cheek region 81 and the forehead region 82 at this time are sizes determined by the region determiner 35.

The blood pressure calculator 53 applies the pulse wave propagation time calculated by the propagation time calculator 52 to the model (graph of the broken line shown in FIG. 7) set by the model setter 36, thereby calculating the blood pressure value. The blood pressure calculator 53 outputs the blood pressure value thus calculated to the display unit 6.

As described above, in the blood pressure measurement device 1, the model setting unit 3 sets an optimal model, based on which the blood pressure measurement unit 5 predicts the blood pressure. In the blood pressure measurement device 1, contactless measurement by using the camera 10 significantly reduces labor of nurses or inconvenience of the blood pressure measurement at home as compared to a conventional cuff system.

Moreover, the contactless measurement by using the camera 10 enables the mental and physical health condition to be grasped without the user's awareness. Thus, health management of a driver driving an automobile and/or elderly people can be appropriately performed.

### Second Embodiment

Another embodiment of the present invention will be described in detail below.

In the first embodiment, the face of a subject is fixed so as not to move with respect to the camera 10, and therefore, the number of pixels in the cheek region 81 and the forehead region 82 is fixed between moving images or between frames of an identical moving image. However, the face of the subject may move in practice.

In this case, a general face recognition algorithm is used to perform facial recognition at intervals of several frames, and in accordance with an actual detection region size, the size of the target region of each part is made variable. Such an image analysis is performed by a region setter 31.

The region setter 31 identifies an image (base image) of the subject present at a reference distance (initial distance), calculates an enlargement ratio or a reduction ratio when the figure of the subject in another image is enlarged or reduced to match the size of the base image, and changes areas of a cheek region 81 and a forehead region 82 serving as references based on the enlargement ratio or the reduction ratio.

Also when the areas of the cheek region 81 and the forehead region 82 are changed between a plurality of moving images, the areas of the cheek region 81 and the forehead region 82 serving as the references in a moving image captured for the second and subsequent times are changed in the same manner based on the initial distance when an initial moving image is captured.

This can be organized as described below. That is, the region setter 31 defines, as the base image, an image in which a subject is present at the reference distance, and the region setter 31 performs a change for each of the plurality of images of the subject such that the areas of the cheek region 81 and the forehead region 82 serving as the references correspond to areas of the cheek region 81 and the forehead region 82 respectively set in the base image.

### Third Embodiment

Still another embodiment of the present disclosure will be described in detail below. Note that for the sake of description, members having the same functions as those described in the embodiments are denoted by the same reference signs, and the description thereof will not be repeated.

A plurality of explanatory variables relating to waveform information correlated with not only the pulse wave propagation time but also the blood pressure are added to a model, thereby improving predicting performance.

FIG. 9 is a view illustrating a method for calculating the waveform information from a pulse waveform in 16×16 pixels of a forehead region 82. The pulse waveform is calculated from a 60-second moving image. Here, valid maximum and minimum of the pulse wave are detected, and pieces of waveform information are pieces of information shown below.

AMP1: Amplitude from a minimum to a next maximum
AMP2: Amplitude from a maximum to a next minimum
T1: Time (T2+T3) from a minimum to a next minimum
T2: Time from a minimum to a next maximum
T3: Time from maximum to a next minimum
T4: Time from a maximum to a next maximum
SLP1: Gradient (AMP1/T2) from a minimum to a next maximum
SLP2: Gradient (AMP2/T3) from a maximum to a next minimum

A blood pressure prediction model is considered in which each piece of waveform information, in addition to the pulse wave propagation time when the target region size is optimal as shown in the first embodiment, is defined as an explanatory variable. For creation of the model, data obtained by simultaneously acquiring a blood pressure value, a pulse wave propagation time, and each piece of waveform information is used. To select a model having high predicting performance, a more complicated model is preferably selected without overfitting under a limited data size.

For example, when a method called Lasso regression is used, a regularization parameter determining the complexity of a model is optimized such that a prediction error is minimum, thereby selecting a blood pressure prediction model with a high accuracy by using a pulse wave propagation time and each piece of waveform information.

The waveform information may be based on a cheek region 81 or based on the forehead region 82. Alternatively, pieces of wave information based on both of the regions may be used.

Lasso regression was performed, where the cheek region 81 has 32×32 pixels, the forehead region 82 has 16×16 pixels, and a pulse wave propagation time between these regions and each piece of waveform information of the forehead region 82 are defined as explanatory variables. Fourteen data sets in total each obtained by simultaneously acquiring a blood pressure value, a pulse wave propagation time, and each piece of waveform information were used.

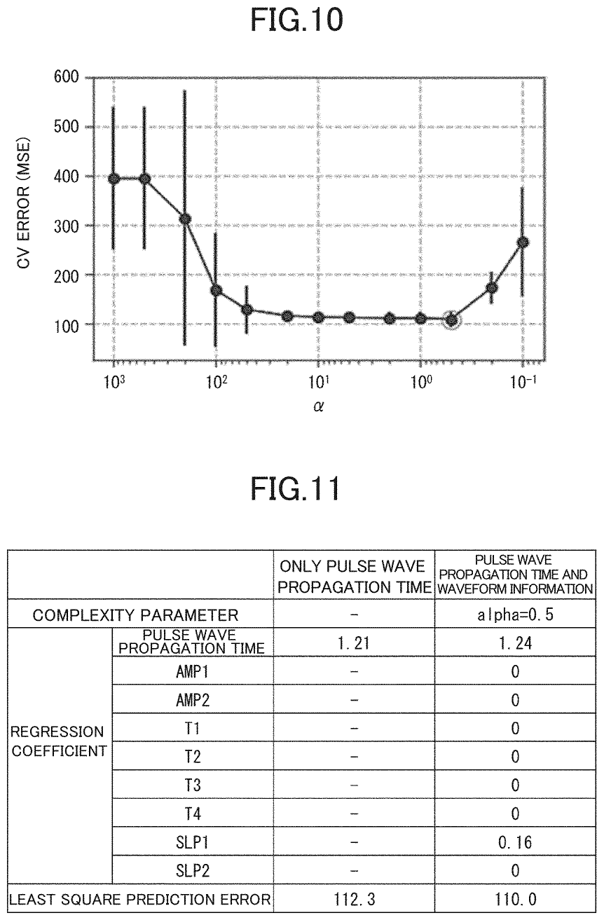

FIG. 10 shows a relationship between a mean squared prediction error (ordinate) and a regularization parameter (abscissa) when Lasso regression was performed. As the value of the regularization parameter decreases, the model complexity increases. From this result, the value of the optimal regularization parameter when the mean squared prediction error is minimum resulted in 0.5.

FIG. 11 is a view illustrating a result of comparison between a case where Lasso regression is performed without using the waveform information and a case where the Lasso regression is performed by using the waveform information (the value of the regularization parameter is 0.5), and FIG. 11 illustrates the coefficient of each explanatory variable and a prediction error. When the waveform information is used, using SLP1 in addition to the pulse wave propagation time reduced the prediction error and improved the prediction accuracy.

Note that in the result at this time, the coefficients of the variables of the pieces of waveform information other than SLP1 were zero, but this is because the number of pieces of data is 14, that is, small, and when the number of the variables is increased and the model is made complicated, over-training occurs. It is said that the number of pieces of data is desirably 5 to 10 times the number of variables, and when the number of pieces of data increases, the prediction error may be further improved in a more complicated model such as a model to which variables other than SLP1 are added.

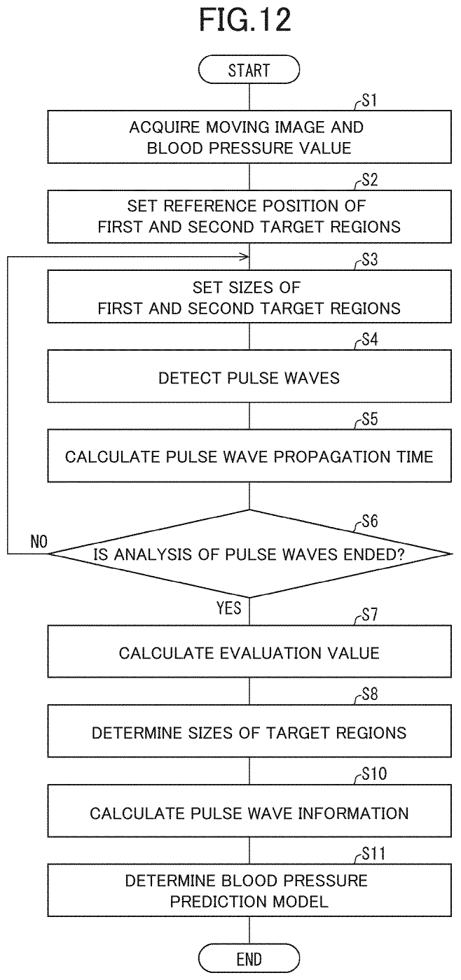

Based on the above-described technical idea, a model setter 36 may set the blood pressure prediction model. FIG. 12 is a flowchart illustrating an example of a flow of a process in a data acquisition unit and a model setting unit according to the present embodiment. Steps of performing the same processes as those in the steps shown in FIG. 3 are denoted by the same reference signs.

As illustrated in FIG. 12, the model setter 36 calculates a feature shown by a waveform of a pulse wave at the first or second target region used for calculation of the pulse wave propagation time by analyzing the waveform (S10). The feature is a value corresponding to the above-described waveform information.

The model setter 36 determines a model with a minimum prediction error based on the blood pressure value of a subject, the pulse wave propagation time between the first target region and second target region corresponding to the blood pressure value, and the waveform information of the pulse wave at the first or second target region used for calculation of the pulse wave propagation time. The model setter 36 determines, by using a prediction error of a model in a plurality of regularization parameters as an index, the most preferable model of a plurality of models obtained from the blood pressure value and the pulse wave propagation time as a model for a blood pressure prediction (S11).

### Example of Actualization by Software

A control block (in particular, the model setting unit 3 and the blood pressure measurement unit 5) of the blood pressure measurement device 1 may be realized by a logic circuit (hardware) formed on an integrated circuit (IC chip) or the like or may be realized by software.

In the latter case, the blood pressure measurement device 1 includes a computer that executes a command of a program (model setting program) which is software realizing each function. The computer includes, for example, at least one processor (a control device) and includes at least one computer-readable recording medium storing the program. In the computer, the processor reads the program from the recording medium and executes the program to achieve the object of the present disclosure. As the processor, for example, a Central Processing Unit (CPU) may be used. As the recording medium, a “non-transitory tangible medium”, for example, in addition to Read Only Memory (ROM), a tape, a disk, a card, semiconductor memory, a programmable logic circuit, or the like may be used. Moreover, Random Access Memory (RAM) and the like in which the program is developed may further be provided. The program may be supplied to the computer over any transmission medium (for example, communication network or broadcast wave) that is capable of transmitting the program. Note that, an aspect of the present disclosure can be embodied also in a form of a data signal in which the program is realized by electronic transmission and which is embedded in a carrier wave.

### Note

The present invention is not limited to the description of the embodiments above and may be altered within the scope of the Claims. Embodiments based on a proper combination of technical means disclosed in different embodiments are encompassed in the technical scope of the present invention. Furthermore, a new technological feature may be created by combining different technological means disclosed in the embodiments.

### CROSS-REFERENCE OF RELATED APPLICATION

The present disclosure contains subject matter related to that disclosed in Japanese Priority Patent Application JP 2018-060592 filed in the Japan Patent Office on Mar. 27, 2018, the entire contents of which are hereby incorporated by reference.

### REFERENCE SIGNS LIST

1 BLOOD PRESSURE MEASUREMENT DEVICE (CONTACTLESS BLOOD PRESSURE MEASUREMENT DEVICE)
3 MODEL SETTING UNIT (MODEL SETTING DEVICE)
5 BLOOD PRESSURE MEASUREMENT UNIT
10 CAMERA
20 SPHYGMOMANOMETER
31 REGION SETTER
32, 51 PULSE WAVE DETECTOR (DETECTOR)
33, 52 PROPAGATION TIME CALCULATOR (CALCULATOR)
34 EVALUATOR
35 REGION DETERMINER (DETERMINER)
36 MODEL SETTER
53 BLOOD PRESSURE CALCULATOR
80 FACE REGION
81, 81A, 81A⋅81B, 81A⋅B, 81B CHEEK REGION
82, 82A, 82A⋅82B, 82A⋅B, 82B FOREHEAD REGION
100 BLOOD PRESSURE MEASUREMENT SYSTEM

1. A model setting device configured to set a model for predicting blood pressure of a living body based on pulse waves at first and second target parts of the living body,
the model setting device being configured to acquiring a plurality of combinations of a blood pressure value of the living body and a plurality of images of the living body when the blood pressure value is measured,
the model setting device comprising:
a region setter configured to perform a process of setting a plurality of first target regions having different sizes in the first target part and setting a plurality of second target regions having different sizes in the second target part on the plurality of images included in each of the plurality of combinations;
a detector configured to detect pulse waves of the living body based on the plurality of first and second target regions thus set;
a calculator configured to calculate a pulse wave propagation time between the first and second target regions from the pulse waves thus detected;
an evaluator configured to evaluate, based on the blood pressure value and the pulse wave propagation time, an accuracy of a blood pressure prediction by using the first and second target regions for each combination of the sizes of the first and second target regions; and
a determiner configured to determine preferable sizes of the first and second target regions based on an evaluation result by the evaluator.

2. The model setting device according to claim 1, wherein
the evaluator evaluates to which degree a relationship between the blood pressure value and the pulse wave propagation time approximates a prescribed relationship, and
the determiner determines that sizes of the first target region and the second target region at which the relationship between the blood pressure value and the pulse wave propagation time is most approximate to the prescribed relationship are the preferable sizes of the first target region and the second target region.

3. The model setting device according to claim 1, further comprising
a model setter configured to set a model corresponding to a combination of the sizes of the first target region and the second target region determined by the determiner as the model for predicting the blood pressure.

4. The model setting device according to claim 3, wherein
the model setter sets the model further based on a feature shown by a waveform of the pulse wave detected by the detector.

5. The model setting device according to claim 1, wherein
the region setter defines an image in which the living body is located at a reference distance as a base image and performs a change for each of the plurality of images of the living body such that areas of the first and second target regions serving as references correspond to areas of the first and second target regions set in the base image.

6. A contactless blood pressure measurement device comprising the model setting device according to claim 1.

7. A non-transitory computer-readable recording medium on which a model setting program for causing a computer to function as the model setting device according to claim 1 is stored, the model setting program being configured to cause the computer to function as the region setter, the detector, the calculator, the evaluator, and the determiner.

8. (canceled)

9. A model setting method for setting a model for predicting blood pressure of a living body based on pulse waves at first and second target parts of the living body, the model setting method comprising:
a step of acquiring a plurality of combinations of a blood pressure value of the living body and a plurality of images of the living body when the blood pressure value is measured;
a step of performing a process of setting a plurality of first target regions having different sizes in the first target part and setting a plurality of second target regions having different sizes in the second target part on the plurality of images included in each of the plurality of combinations;
a step of detecting pulse waves of the living body based on the plurality of first and second target regions thus set;
a step of calculating a pulse wave propagation time between the first and second target regions from the pulse waves thus detected;
a step of evaluating, based on the blood pressure value and the pulse wave propagation time, an accuracy of a blood pressure prediction by using the first and second target regions for each combination of the sizes of the first and second target regions; and
a step of determining preferable sizes of the first and second target regions based on the evaluation.

10. The model setting device according to claim 2, further comprising
a model setter configured to set a model corresponding to a combination of the sizes of the first target region and the second target region determined by the determiner as the model for predicting the blood pressure.

11. The model setting device according to claim 10, wherein
the model setter sets the model further based on a feature shown by a waveform of the pulse wave detected by the detector.

12. The model setting device according to claim 2, wherein
the region setter defines an image in which the living body is located at a reference distance as a base image and performs a change for each of the plurality of images of the living body such that areas of the first and second target regions serving as references correspond to areas of the first and second target regions set in the base image.

13. The model setting device according to claim 3, wherein
the region setter defines an image in which the living body is located at a reference distance as a base image and performs a change for each of the plurality of images of the living body such that areas of the first and second target regions serving as references correspond to areas of the first and second target regions set in the base image.

14. The model setting device according to claim 10, wherein
the region setter defines an image in which the living body is located at a reference distance as a base image and performs a change for each of the plurality of images of the living body such that areas of the first and second target regions serving as references correspond to areas of the first and second target regions set in the base image.

15. The model setting device according to claim 4, wherein
the region setter defines an image in which the living body is located at a reference distance as a base image and performs a change for each of the plurality of images of the living body such that areas of the first and second target regions serving as references correspond to areas of the first and second target regions set in the base image.

16. The model setting device according to claim 11, wherein
the region setter defines an image in which the living body is located at a reference distance as a base image and performs a change for each of the plurality of images of the living body such that areas of the first and second target regions serving as references correspond to areas of the first and second target regions set in the base image.
